(19) United States
(12) Patent Application Publication
(10) Pub. No.: US 2017/0038335 A1
(43) Pub. Date: Feb. 9, 2017

(54) FRAME MEMBER-EQUIPPED TRANSFER FILM, BIOMOLECULE ANALYSIS DEVICE, REAGENT TANK, AND SHAKING DEVICE

(71) Applicant: SHARP KABUSHIKI KAISHA, Sakai City (JP)

(72) Inventors: Mieko HIRABAYASHI, Sakai City (JP); Hideki KINOSHITA, Sakai City (JP); Shinichi GOTO, Sakai City (JP); Taiga TOMINAGA, Sakai City (JP); Kouhei KAGEYAMA, Sakai City (JP)

(21) Appl. No.: 15/303,553

(22) PCT Filed: Nov. 18, 2015
(86) PCT No.: PCT/JP2015/082345

§ 371 Date: Oct. 12, 2016

(30) Foreign Application Priority Data
Nov. 20, 2014 (JP) 2014-235990

(51) Int. Cl. G01N 27/447; G01N 33/543

(52) U.S. Cl. CPC G01N 27/44704; G01N 27/44778; G01N 33/54366

(57) ABSTRACT

A frame member-equipped transfer film (10) is provided with a transfer film (1) to which a sample separated by electrophoresis is transferred, and a pair of frames (2) that individually support two sides of the transfer film (1), namely a first side of the transfer film (1) and a second side opposite the first side.

### TECHNICAL FIELD

The present invention relates to a transfer membrane with frame member, a biomolecule analyzer, a reagent tank and a shaker device.

### BACKGROUND ART

In proteome analysis, which bears a central position in post-genomic research, a combination of two-dimensional electrophoresis (2DE) and Western blotting has been known as an excellent separation analytical method. 2DE can separate proteomes with high resolution into a plurality of components (proteins), based on two independent physical properties unique to proteins (electric charge and molecular weight) using various separation media. In the case of further analyzing proteins using the separation results by 2DE, it is preferable to fixate the plurality of proteins contained in the separation medium on a transfer membrane by way of the Western blotting. This is because analysis is easy, since the proteins fixed to the transfer membrane can be stably kept over a long period. In particular, in the case of comprehensively comparing and reviewing a plurality of biological properties of proteins like the fluctuations in expression of proteins and existence of post-translational modification using the separation results by 2DE, Western blotting is considered an essential process.

In the case of the conventional, known techniques of performing 2DE and Western blotting, respectively, using independent devices, after electrophoresis, operations are necessary for retrieving the separation medium from the electrophoresis device, moving to a transfer device, setting a transfer membrane therein, and performing transfer.

At this time, in order to obtain favorable results, it is preferable to perform transfer in a state tightening the transfer membrane without slack. Patent Document 1 describes fixing the transfer membrane in a curved frame in a state without wrinkles in the transfer membrane, transferring proteins thereto by way of an electrophoresis device, followed by placing the transfer membrane mounted to the curved frame into liquid chamber for every frame, and performing liquid treatment.

Patent Document 1: Japanese Published Translation of PCT International Publication for Patent Applications “Japanese Unexamined Patent Application (Translation of PCT Publication), Publication No. H9-501239 (published Feb. 4, 1997)”

### DISCLOSURE OF THE INVENTION

### Problems to be Solved by the Invention

However, with the technology of Patent Document 1, the transfer membrane is mounted to a frame made of resin with two sides curved, and enters a state in which the transfer membrane is always tightened without slack by this frame. For this reason, in the case of storing for a long time, there is the risk of the transfer membrane stretching from the tension applied by the frame, or the tension applied on the transfer membrane varying by the frame being immobilized in a curved state.

In addition, when processing the transfer membrane after transferring proteins by Western blot or the like onto the transfer membrane, it is necessary to enlarge the reagent tank for processing the transfer membrane, and thus there is a problem in that the amount used of reagent becomes great.

In addition, due to structures like that fixing the transfer membrane in the curved frame, even when immersing in reagent and shaking the reagent tank, there is a risk of not being able to spread the sample on the surface of the transfer membrane uniformly.

The present invention has been made by taking account of the above-mentioned problems, and a main object thereof is to provide technology for easily tightening the transfer membrane without slack, and without inhibiting the treatment after transfer.

### Means for Solving the Problems

In order to solve the above-mentioned problems, a transfer membrane with frame member according to one aspect of the present invention includes: a transfer membrane to which an analyte separated by way of electrophoresis is transferred, and a pair of frame members that individually support two sides, which are a first side that is one side of the transfer membrane, and a second side that is a side thereof on a side opposing the first side.

### Effects of the Invention

According to an aspect of the present invention, it is possible to easily tighten a transfer membrane without slack, and not inhibiting processing after transfer.

### BRIEF DESCRIPTION OF THE DRAWINGS

FIG. 1 is a view illustrating an outline of a framed transfer membrane according to a first embodiment of the present invention;

FIG. 2 is a view illustrating an outline of a framed transfer membrane according to a second embodiment of the present invention;

FIG. 3 is a view illustrating an outline of a framed transfer membrane according to a third embodiment of the present invention;

FIG. 4 is a view illustrating an outline of the frame of a transfer membrane according to a modified example of the present invention;

FIG. 5 is a view illustrating an outline of a state mounting the framed transfer membrane to a biomolecule analyzer according to an embodiment of the present invention;

FIG. 6 is a view illustrating an outline of a biomolecule analyzer according to a fifth embodiment of the present invention;

FIG. 7 is a view illustrating an outline of a shaker device according to an embodiment of the present invention;

FIG. 8 is a view illustrating an outline of a shaker device according to a modified example of the present invention;

FIG. 9 is a view illustrating an outline of a framed transfer membrane according to the fourth embodiment of the present invention;

FIG. 10 is a view illustrating an outline of a retaining unit for retaining the framed transfer membrane according to an embodiment of the present invention; and

FIG. 11 is a view illustrating an outline of a biomolecule analyzer according to a sixth embodiment of the present invention.

### PREFERRED MODE FOR CARRYING OUT THE INVENTION

### <Framed Transfer Membrane>

### First Embodiment

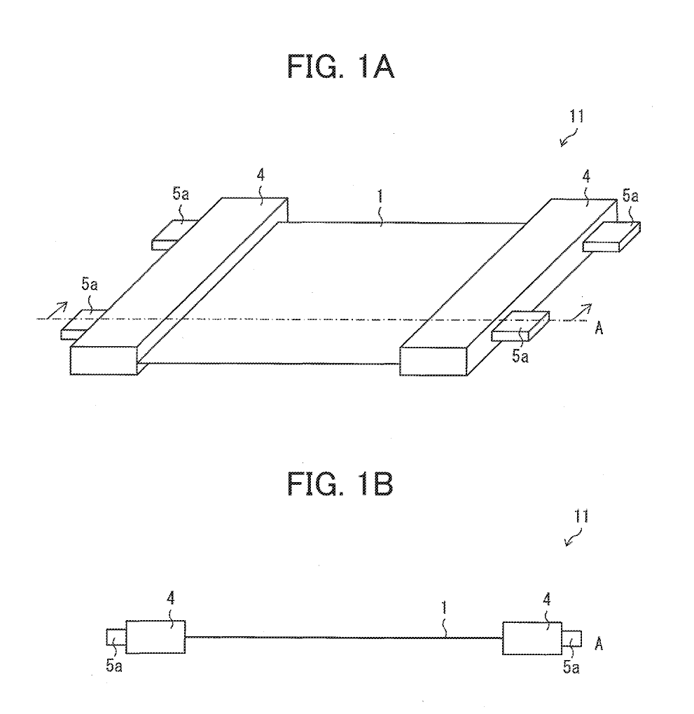

An embodiment (first embodiment) of the present invention will be explained in detail using FIG. 1. FIG. 1(a) is a view illustrating an outline of a framed transfer membrane (transfer membrane with frame member) 10 according to the first embodiment of the present invention. FIG. 1(b) provides cross-sectional views along lines A and B in FIG. 1(a).

As shown in FIG. 1(a), the framed transfer membrane 10 according to the present embodiment is supported with one side of the transfer membrane 1 (first side) and one side opposing this side (second side) independently by a pair of frames (frame members) 2.

As shown in the cross-sectional views on line A and line B in FIG. 1(b), the framed transfer membrane 10 more preferably is provided with fitting parts (through holes) of open form through which the frame 2 passes, as fitting parts 3a of the pair of frames 2. The fitting parts 3a can each fit to fitting parts of a corresponding convex-type shape. It is thereby possible to fix the framed transfer membrane 10 and biomolecule analyzer with the fitting parts 3a.

The framed transfer membrane 10 can tighten the transfer membrane 1 without slack by horizontally fixing the pair of frames 2 to be separated from each other. In addition, since it is possible to fix the transfer membrane 1 which is sectioned into a predetermined shape in advance by the frame 2, the framed transfer membrane 10 can be installed in the biomolecule analyzer while imparting easy and appropriate tension to the transfer membrane 1. For this reason, it is possible to prevent warping of the blot when fixing to the biomolecule analyzer and transferring analyte by direct blotting.

In addition, with the framed transfer membrane 10, it is possible to use the frame 2 as a sinker of the transfer membrane 1. For this reason, it is possible to prevent the transfer membrane 1 in the reagent from moving inside a reagent tank such as a shaker, and irregularity arising in the antibody response. Furthermore, the framed transfer membrane 10 is a simple configuration consisting of the transfer membrane 1 and pair of frames 2; therefore, compared with one that broadly fixes the transfer membrane smoothly by fixing the entire periphery by a frame, and one that broadly fixes the transfer membrane by a curved frame, it is not bulky and, for example, it is possible to reduce the amount of antibody used when performing Western blotting.

### (Transfer Membrane 1)

The transfer membrane 1 is a membrane for adsorbing and retaining biomolecular sample separated by the separation unit of the biomolecule analyzer. Herein, it is preferable for the transfer membrane 1 to be able to stably preserve a biomolecular sample (analyte) separated by the separation unit over a long period, and further, and to be an absorbing/retaining body of biomolecular samples that facilitates subsequent analysis. As the material of the transfer membrane 1, it is preferably a material having high strength, and having high sample binding capacity (adsorbable weight per unit volume). As the transfer membrane 1, a polyvinylidene fluoride (PVDF) membrane or the like is suited in the case of the sample being protein. It should be noted that it is preferable to perform hydrophilization treatment using methanol or the like in advance on the PVDF membrane. Otherwise, a membrane conventionally used in the adsorption of proteins, DNA and nucleic acids such as a nitrocellulose membrane or nylon membrane can also be used.

It should be noted that, as the biomolecular samples (analyte) that can be separated and adsorbed in the biomolecule analyzer, although proteins, DNA and RNA can be exemplified, it is not limited thereto. For example, a preparation from biological material (e.g., cell strain, tissue culture, or tissue fragment), a commercially available reagent, and the like can be exemplified. For example, polypeptides or polynucleotides can be exemplified.

### (Frame 2)

For each of the pair of frames 2, the length of the frame 2 is sufficient if longer than the length of one side of the transfer membrane 1 to be fixed. In addition, the frame 2 preferably consists of an insulating material. As the insulating material, it is possible to use a resin such as polymethylmethacrylate (acrylic), polystyrene, polyethylene, polypropylene, polyethylene terephthalate (PET), polyacetal (POM) and polyether ether ketone (PEEK), or glass.

In addition, hydrophilization treatment is more preferably conducted on the surface of the frame 2. For example, a surface coating layer formed by a hydrophilic material, for example, may be provided to the surface of the frame 2 consisting of the above-mentioned materials. In addition, hydrophilicity may be imparted to the frame 2 by using a hydrophilic material such as glass as the frame 2. It is thereby possible to prevent a reagent such as protein dispensed from the dispensing part of the electrophoresis gel chip 50 from adhering to the surface of the frame 2, and possible to prevent the frame 2 from being contaminated.

It should be noted that the water contact angle to the surface of the frame 2 is preferably no more than 90°, and more preferably no more than 60°. By establishing the water contact angle to the surface of the frame 2 as no more than 90°, it is possible to suitably prevent the frame 2 from being contaminated by reagent.

### Second Embodiment

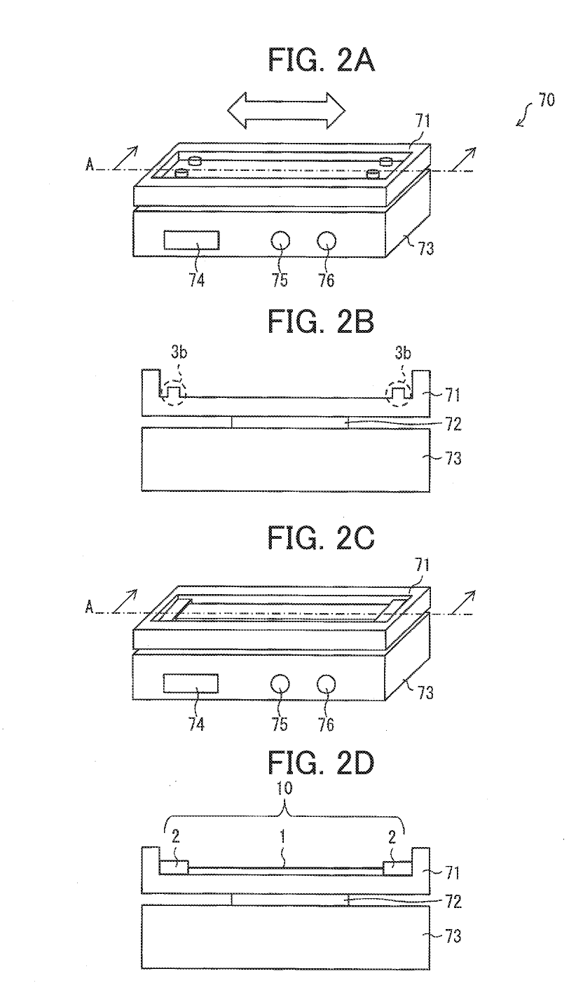

The framed transfer membrane according to the present invention is not limited to the first embodiment described above. As shown in FIGS. 2(a) and (b), for example, with the framed transfer membrane 11 according to an embodiment (second embodiment), the transfer membrane 1 is supported by a pair of frames 4 equipped with convex fitting parts 5a (projecting parts). FIG. 2(a) is a perspective view showing the framed transfer membrane 11 according to the present embodiment, and FIG. 2(b) is a cross-sectional view along the line A of the framed transfer membrane 11 according to the present embodiment shown in FIG. 2(a).

With the framed transfer membrane 11, it is thereby possible to fix the pair of frames 4 in parallel, and by separating from each other, easily tighten the transfer membrane 1 itself without slack, similarly to the case of the framed transfer membrane 10. For this reason, the framed transfer membrane 11 can prevent warping of the blot when transferring analyte by direct blotting, and it is possible to use the frame 4 as a sinker of the transfer membrane 1.

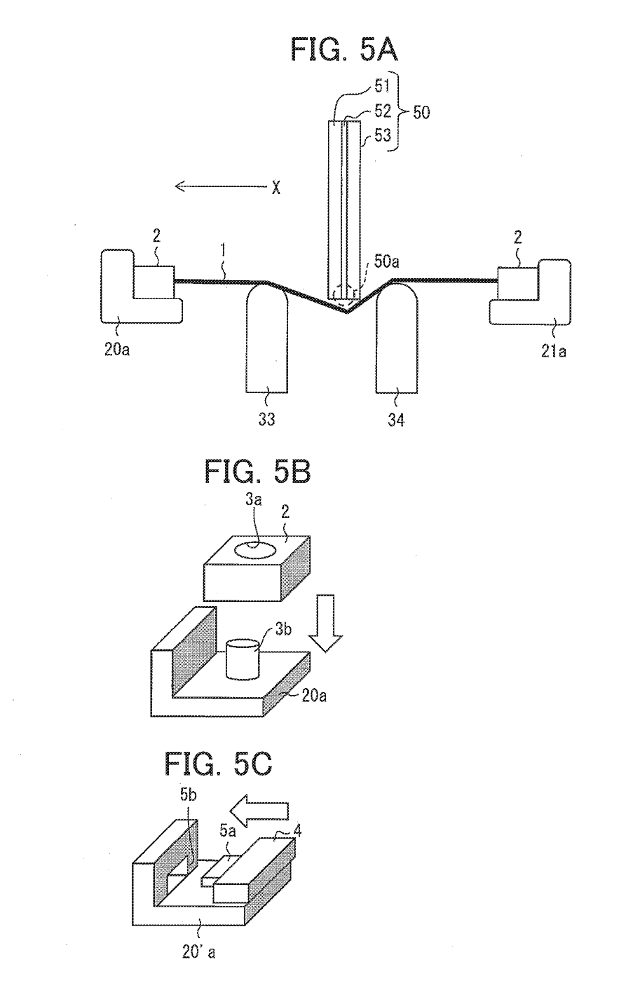

In addition, as shown in FIG. 5(c), with the framed transfer membrane 11, the fitting part 5a provided to one of the frames 4 is fitted in a concave fitting part 5b provided to a forward clamp 20′a. In addition, it is possible to fix the fitting part 5a provided to the other frame 4 in the fitting part provided to the rear clamp 21a. It should be noted that the fitting part 5b corresponding to the fitting part 5a of the framed transfer membrane 11 according to the present embodiment more preferably is a structure that can retain by inserting the convex part of the fitting part 5a as in a Duracon catch, or the like.

### Third Embodiment

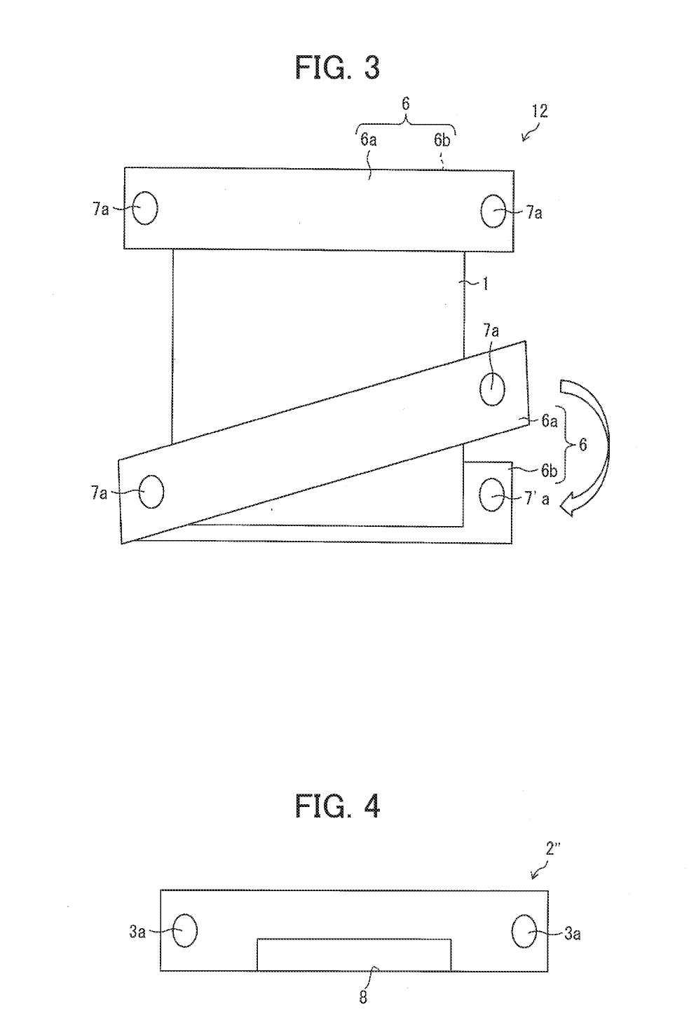

The framed transfer membrane according to the present invention is not limited to the above-mentioned first embodiment. FIG. 3 is a view illustrating an outline of a framed transfer membrane 12 according to one embodiment of the present invention. As shown in FIG. 3, for example, with the framed transfer membrane 12 according to one embodiment (third embodiment), the transfer membrane 1 is supported at one side thereof and a side that is on a side opposing this side by the frames 6, respectively.

The frames 6 support the transfer membrane 1 by sandwiching the two opposing sides of the transfer membrane 1, on faces at which the frame 6a and frame 6b are opposing. At faces on the sides at which the frame 6a and frame 6b are opposing each other, guides matching the shape of a portion in which the transfer membrane is sandwiched may be provided so as to be able to arrange the transfer membrane 1 at a predetermined position. In addition, the frame 6a and frame 6b can be fixed by the fitting parts (not illustrated) to each other. It should be noted that, when fixing the frame 6a and frame 6b, the shapes of the openings match between fitting part 7a and fitting part 7′a, and thereby are configured to be able to fit with the corresponding convex fitting parts.

Also according to the above-mentioned configuration, similarly to the case of the framed transfer membrane 10 and framed transfer membrane 11, it is easily possible to evenly expand the transfer membrane 1 itself, by fixing the pair of frames 6 in a state expanding evenly the transfer membrane 1 suitably in parallel to separate from each other.

In addition, according to the above-mentioned configuration, it becomes possible to replace after evaluating the transfer membrane 1 to which the analyte is transferred by the biomolecule analyzer, and thus is becomes possible to reuse the frame member.

### Fourth Embodiment

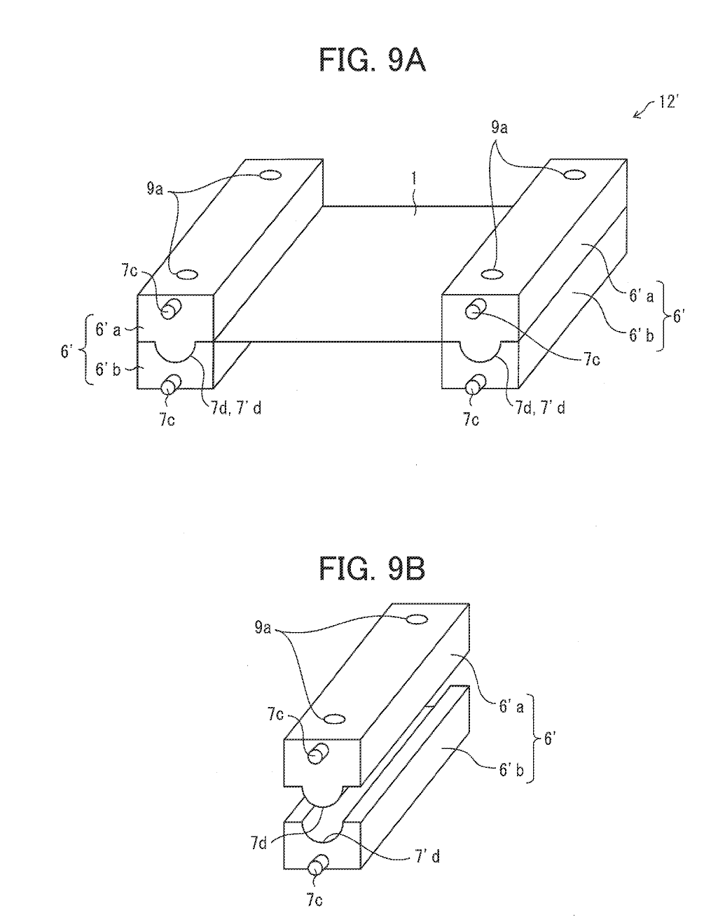

The framed transfer membrane according to the present invention is not limited to the above-mentioned first embodiment. FIGS. 9(a) and (b) are views illustrating outlines of a framed transfer membrane 12′ according to one embodiment (fourth embodiment) of the present invention.

As shown in FIG. 9(a), for example, the framed transfer membrane 12′ according to one embodiment (fourth embodiment) includes a first face and second face forming a pair that sandwich the transfer membrane 1, respectively, and a ridge part 7d having a contour in which a cross section in a short direction has a curved shape is provided to the first face, and a grooved part 7′d fitting with the ridge part 7d is provided to the second face. Herein, the ridge part 7d of the frame 6′a and the grooved part 7′d of the frame 6′b can be fitted together. As shown in FIG. 9(a), the frame 6′ can thereby support the transfer membrane 1 by sandwiching between the ridge part 7d of the frame 6′a and the grooved part 7′d of the frame 6′b, which can raise the frictional force of the faces sandwiching the transfer membrane 1 of the frame 6′. For this reason, in the biomolecule analyzer, it is possible to more suitably prevent from the transfer membrane 1 detaching from the frame 6′, when sandwiching the transfer membrane 1 by the pair of frames 6′ and conveying the transfer membrane 1. It should be noted that the ridge part 7d provided to the frame 6′a is more preferably formed by a material, other than the resin constituting the frame, having high insulation property and friction property such as an elastic member like rubber or a ceramic. So long as forming the ridge part 7d having the contour of a cross-section in the short direction of the frame 6′ that is a curved line by an elastic member like rubber or a ceramic, it is possible to raise the frictional force of the ridge part 7d and grooved part 7′d, and thus possible to more suitably prevent the transfer membrane 1 from detaching from the frame 6′.

In addition, as shown in FIGS. 9(a) and (b), convex parts 7c are provided to both ends in the length direction of the frame 6′a and the frame 6′b, respectively. By the frame 6′ fitting the convex parts 7c provided to each into a jig (not illustrated) equipped with concave parts that can fit with the convex parts 7c, it is possible to fix the frame 6′a and frame 6′b in a state sandwiching the transfer membrane 1. In addition, the framed transfer membrane 12′ can fix to the biomolecule analyzer by causing to fit the fitting part 9a with the corresponding fitting part.

### Modified Example

The framed transfer membrane according to the present invention is not limited to the above-mentioned embodiment. FIG. 4 is a view illustrating an outline of a frame of the transfer membrane according to a modified example of the present invention. As shown in FIG. 4, with the framed transfer membrane according to a modified example, for example, a modified region 8 that can be modified by a fluorescence reference sample in advance is provided to a frame 2″. Herein, the modified region 8 may be modified by a predetermined amount of the fluorescence reference sample.

It is thereby possible to adjust the intensity of light exposed by the fluorescent scanner, for example, based on the fluorescence intensity emitted by the fluorescence reference sample modifying the modified region 8. It should be noted that the modified region 8 may be provided to both of the pair of frame members, or may be provided to only either one.

### Other Modified Example

The framed transfer membrane according to the present invention is not limited to the above-mentioned embodiment. For example, with the framed transfer membrane according to another modified example, an entry region enabling the entry of information is provided to the pair of frames (frame member).

It is thereby possible to record various information such as the type of analyte, evaluation conditions, date transferring analyte to the transfer membrane, and time, for every framed transfer membrane.

### Fifth Embodiment

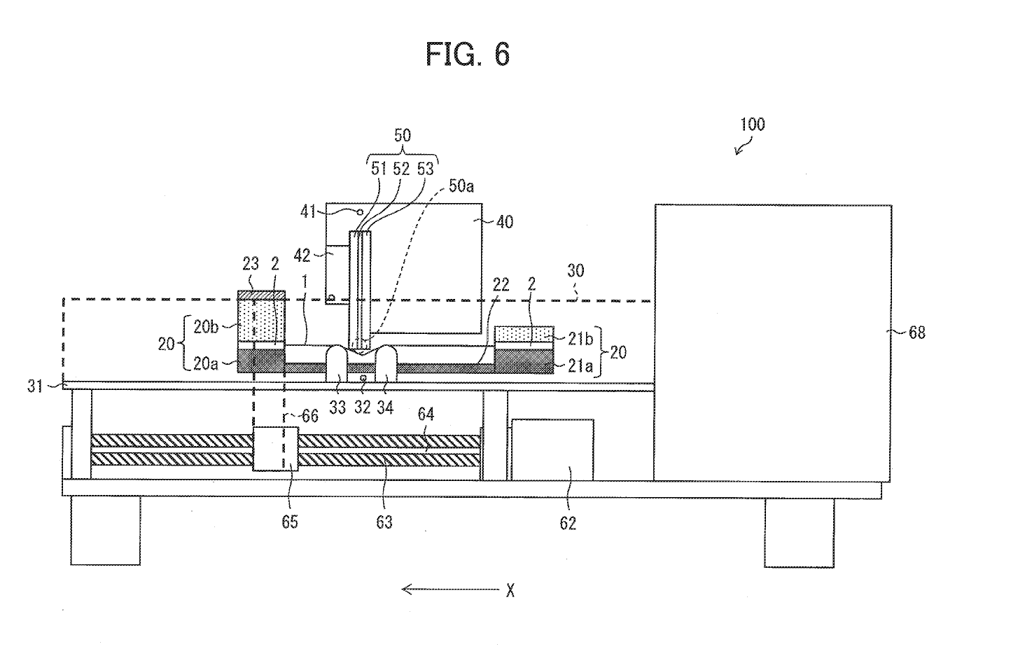

A biomolecule analyzer according to one embodiment (fifth embodiment) of the present invention will be explained in further detail using FIG. 6. FIG. 6 is a view illustrating an outline of a biomolecule analyzer 100 according to the fifth embodiment of the present invention.

The biomolecule analyzer 100 according to the present embodiment is configured from a clamp 20 fixing the framed transfer membrane 100, an anode buffer tank 30, a cathode buffer tank 40, an electrophoresis gel chip 50 and a conveying unit consisting of a motor 62, etc.

The electrophoresis gel chip 50 separates the analyte by way of electrophoresis, and dispenses the separated analyte onto the transfer membrane 1. The conveying unit conveys the transfer membrane 1 in a conveying direction (direction from one side (first side) of the framed transfer membrane 10 on which the frame 2 is provided, towards another side (second side) on which the frame 2 is provided). The dispensed analyte is thereby adhered at positions on the transfer membrane 1 according to the timing of being dispensed (positions at which opposing the electrophoresis gel chip 50 at the timing of being dispensed). The separated analyte is thereby transferred to the transfer membrane 1.

### (Clamp 20)

As shown in FIG. 6, the biomolecule analyzer 100 according to the present embodiment has a clamp (retaining part) 20 made by fixing the front clamp 20a and rear clamp 21a by a clamp frame 22. Herein, a carrier 23 is provided to the front clamp 20b.

The clamp 20 is fixed so that the front clamp 20a and front clamp 20b can release from the jig, and the fitting parts 3b like that shown in FIG. 5 are provided at two locations to the front clamp 20a. The fitting parts 3a provided at two locations on the frame 2 shown in FIG. 1(a) are fitted to these fitting parts 3b, and subsequently, one of the frames 2 is fixed by sandwiching by the front clamp 20a and front clamp 20b.

Similarly, the rear clamp 21a and the rear clamp 21b are fixed so as to be able to release from the jig, and fitting parts 3b like that shown in FIG. 5(b) are provided at two locations to the rear clamp 21a. It is thereby possible to fix the remaining one of the frames 2.

The clamp frame 22 fixes the front clamp 20a and rear clamp 21a in a state separated by a certain distance. For this reason, when fixing the framed transfer membrane 10 by way of the clamp 20, the transfer membrane 1 is fixed in a tightened state without slack. In addition, when fixing the framed transfer membrane 10 by the clamp, the clamp frame 22 fixes the front clamp 20a and rear clamp 21a from laterals sides relative to the conveying direction of the transfer membrane 1 (outer sides of two sides not supported by the frame 2). When fixing the framed transfer membrane 10 via the clamp 20 to the biomolecule analyzer 100, it is thereby possible to arrange so that the clamp frame 22 does not contact the electrophoresis gel chip 50, or the guides 33 and 34, in the conveying path of the transfer membrane 1.

The carrier 23 is provided to the front clamp 20b, and when installing the clamp 20 to an inner side of the anode buffer tank 30, it is configured so as to be able to fix in a detachable state to the guide poles 66 arranged at the exterior of the anode buffer tank 30.

### (Anode Buffer Tank 30)

The anode buffer tank 30 shown by the dotted line in FIG. 6 is fixed in a detachable state to the anode stage 31. In addition, the anode 32, and guides (support member) 33 and 34 are provided to a bottom part of the anode buffer tank 30. The guides 33 and 34 are support members each supporting, from the opposite side of the transfer membrane 1 than the electrophoresis gel chip 50, a pair of positions interposing, from the front and rear in the conveying direction, a position of the transfer membrane 1 abutted by the electrophoresis gel chip 50.

The anode buffer tank 30 is filled with anode buffer, and is configured so as to be able to install the framed transfer membrane 10 fixed to the clamp 20 inside the anode buffer.

The anode 32 is an electrode constituted by platinum wire, etc., and is provided at the bottom part of the anode buffer tank 30 along a vertical direction on the conveying path on which the framed transfer membrane 10 is conveyed. When the framed transfer membrane 10 is installed, the anode 32 is thereby configured so as to be able to apply voltage between t and the cathode 41 from a back surface on a side opposing the electrophoresis gel chip 50 of the transfer membrane 1.

The guides (support members) 33 and 34 are provided on the conveying path on which the framed transfer membrane 10 is conveyed, at the bottom part of the anode buffer tank 30. The guides 33 and 34 are parallel to the electrophoresis gel chip 50, and are arranged so as to vertically intersect the conveying direction (X direction) on which the framed transfer membrane 10 is conveyed. The guides 33 and 34 thereby support the transfer membrane 1 from a back surface on a side opposing the electrophoresis gel chip 50 of the transfer membrane 1, in parallel to the electrophoresis gel chip 50.

It should be noted that, as the anode buffer, for example, it is possible to use buffer solutions such as a Tris/glycine-based buffer solution, acetic acid buffer solution, sodium carbonate-based buffer solution, CPS buffer solution, Tris/boric acid/EDTA buffer solution, Tris/acetic acid/EDTA buffer solution, MOPS, phosphoric acid buffer solution, and Tris/tricine-based buffer solution.

### (Cathode Buffer Tank 40)

The cathode buffer tank 40 is fixed to the anode buffer tank 30 in a detachable state. In addition, the cathode 41 and lock 42 are provided to the cathode buffer tank 40.

Cathode buffer is filled into the cathode buffer tank 40, and the cathode 41 is installed in the cathode buffer so as to be parallel to the anode 32, which is installed in the anode buffer tank 30. In addition, it is possible to fix the electrophoresis gel chip 50 to be parallel relative to the cathode 41 by the lock 42, which is provided in the cathode buffer tank 40. Herein, the electrophoresis gel chip 50 is configured so that an end on the opposite side to the dispensing part 50a thereof can be immersed in the cathode buffer filling the cathode buffer tank 40. It should be noted that it is possible to use the same buffer solution as the anode buffer in the cathode buffer.

The cathode 41 is an electrode constituted from platinum wire or the like, and is installed so as to be parallel to the anode 32 at an inner side of the cathode buffer tank 40. A voltage is thereby applied between the anode 32 and cathode 41, which are parallel to each other.

### (Electrophoresis Gel Chip 50)

In the electrophoresis gel chip (separation unit) 50, the separation gel 52 is formed between an insulating plate 51 and insulating plate 53. The insulating plate 51 and insulating plate 53, for example, are formed by plates consisting of insulators such as glass and acrylic. The separation gel 52 is a gel for separating the introduced biomolecule sample (analyte) according to molecular weight. As examples of the separation gel 52, polyacrylamide gel, agarose gel and the like are exemplified, and it is preferable to use a gel made to match the buffer solution in the aforementioned suitable compositions. The separation gel 52 can form by filling into the electrophoresis gel chip 50 prior to installing the electrophoresis gel chip 50 to the cathode buffer tank 40. The electrophoresis gel chip 50 opposes the dispensing part 50a, and supplies the biomolecule sample components from the end thereof arranged in the cathode buffer tank 40.

### (Conveying Unit)

The conveying unit includes the motor 62, ball screw 63, guide shaft 64, shaft holder 65 and guide poles 66.

The conveying unit can move the shaft holder 65 in the X direction along the guide shaft 64, by causing the ball screw 63 to rotate by way of the motor 62. The guide poles 66 are fixed to the shaft holder 65, the guide poles 66 support the carrier 23 provided to the clamp 20 from the outer side of the anode buffer tank 30.

The conveying unit causes the framed transfer membrane 10 arranged inside of the anode buffer tank 30 to move in the X direction via the guide poles 66 arranged at the outside of the anode buffer tank 30, by causing the motor 62 to move according to the above-mentioned configuration.

### (Operation of Biomolecule Analyzer 100)

An outline of a state mounting the framed transfer membrane 10 to the biomolecule analyzer will be explained using FIGS. 5(a) and (b). FIG. 5(a) is a view illustrating a state mounting the framed transfer membrane 10 to the conveying unit of the biomolecule analyzer. As shown in FIG. 5(b), the framed transfer membrane 10 fixes one of the frames 2 to the front clamp 20a, by causing the fitting part 3a of the frame 2 to fit with the fitting part 3b provided to the front clamp 20a. In addition, in a similar way, it is fixed by causing the fitting part 3a of the other frame 2 to fit with the rear clamp 21a. Subsequently, as shown in FIG. 6, the framed transfer membrane 10 is fixed by the clamp 20, and arranged at the inner side of the anode buffer tank 30 filling with anode buffer. Herein, the transfer membrane 1 of the framed transfer membrane 10 is fixed in a state supported by the guide 33 and guide 34.

In addition, as shown in FIG. 5(a), the transfer membrane 1 of the framed transfer membrane 10 is supported from a lower side by the guide 33 and guide 34.

Subsequently, the cathode buffer tank 40 to which the electrophoresis gel chip 50 is fixed by the lock 42 is fixed at a top part of the anode buffer tank 30. Herein, the electrophoresis gel chip 50 is installed in a state such that is it pushed from a top side. In other words, the transfer membrane 1 is fixed in a state bent so as to become convex (valley folded shape) to an opposite side to the electrophoresis gel chip 50, by contacting the guide 33, guide 34 and electrophoresis gel chip 50.

Next, while being conveyed in the X direction shown in FIG. 5(a), in a state still pushing against the dispensing part of the electrophoresis gel chip 50, the transfer membrane 1 of the framed transfer membrane 10 in the anode buffer is transferred the analyte dispensed by the electrophoresis gel chip 50 by applying voltage between the anode 32 and cathode 41. For this reason, the tension generated when the transfer membrane 1 is conveyed concentrates on the dispensing part provided at the end of the electrophoresis gel chip 50. In other words, the transfer membrane 1 is conveyed in the X direction, while pushing against the dispensing part of the electrophoresis gel chip 50 with a constant force.

For this reason, with the framed transfer membrane 10, it is possible to prevent an interval forming between the transfer membrane 1 and the dispensing part of analyte of the electrophoresis gel chip 50 when conveying the transfer membrane 1. Therefore, it is possible to suppress the analyte dispensed from the dispensing part of the electrophoresis gel chip 50 from scattering within the anode buffer prior to being transferred to the transfer membrane 1. It is thereby possible to reduce the fluctuation in the bands of analyte transferred to the transfer membrane 1, and it is possible to improve the sensitivity of the biomolecule analyzer.

It should be noted that the front clamp 20a and rear clamp 21a move in the X direction by operating together so as to maintain a certain distance by the clamp frame 22. It is thereby possible to make the tension acting on the transfer membrane 1 being conveyed accompanying movement of the clamp 20 constant. Therefore, it is possible to more suitably transfer the analyte to the transfer membrane 1 without blurring.

In addition, the tension acting on the transfer membrane when conveying the framed transfer membrane is preferably a tension in the range of 0.1 N to 20 N, and most preferably on the order of 6 N, for example. The tension acting on the transfer membrane is a tension within the above-mentioned range, it will be possible to transfer the analyte dispensed from the dispensing part of the separation unit to the transfer membrane with good sensitivity, and thus it will be possible to prevent the transfer membrane from being damaged by excessive tension.

### Sixth Embodiment

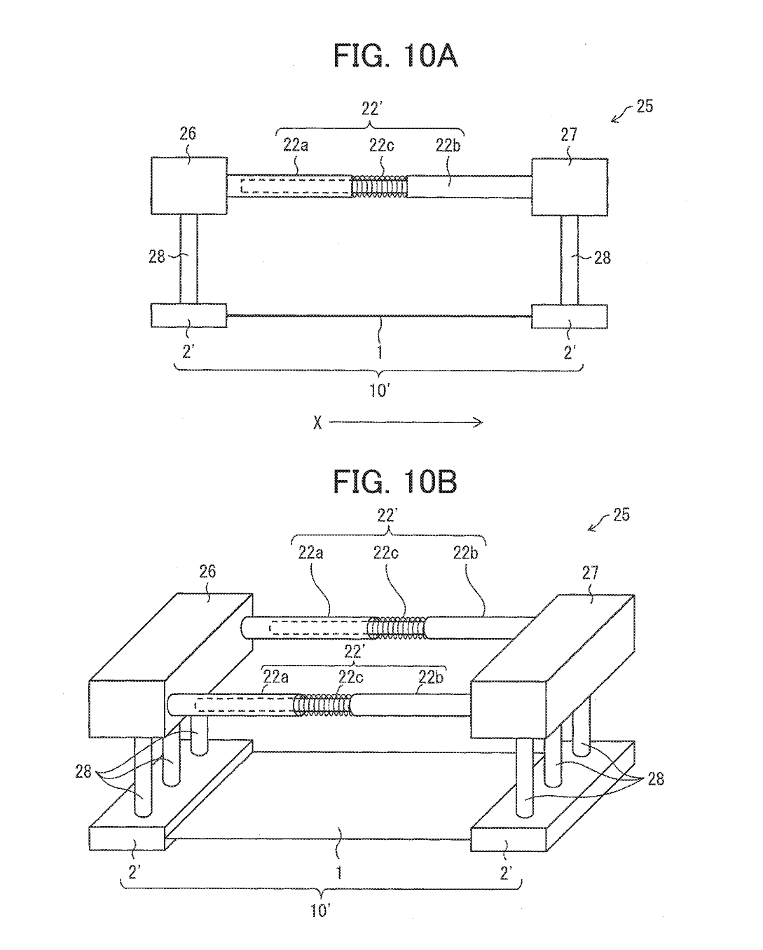

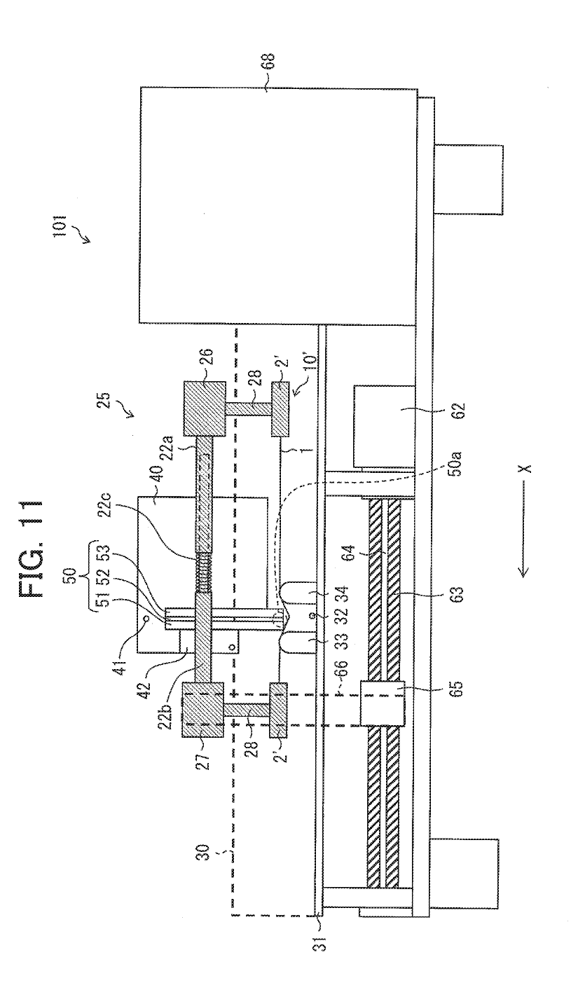

A biomolecule analyzer 101 according to one embodiment (sixth embodiment) of the present invention will be explained in further detail using FIGS. 10 and 11. FIGS. 10(a) and (b) are views illustrating outlines of a clamp (retaining part) 25 that fixes the framed transfer membrane 10′ according to one embodiment of the present invention, and FIG. 11 is a view illustrating an outline of the biomolecule analyzer 101 according to the sixth embodiment of the present invention.

As shown in FIG. 11, the biomolecule analyzer 101 according the present embodiment is configured from the clamp 25 fixing the framed transfer membrane 10′, the anode buffer tank 30, the cathode buffer tank 40, the electrophoresis gel chip 50, and the conveying unit consisting of the motor 62, etc.

It should be noted that, since the anode buffer tank 30, cathode buffer tank 40, electrophoresis gel chip 50, and conveying unit consisting of the motor 62, etc. are the same as for the biomolecule analyzer 100, explanations thereof will be omitted.

### (Clamp 25)

As shown in FIGS. 10(a) and (b), the biomolecule analyzer 101 according to the present embodiment has the clamp (retaining part) 25 made by fixing the front clamp 27 and rear clamp 26 by the clamp frame (fixing part) 22′. Herein, the clamp frame 22′ is configured by the clamp frame 22a, clamp frame 22b and spring (biasing part) 22c, and in the clamp frame 22′, the clamp frame 22a having an inner side of the frame that is hollow, and the clamp frame 22b inserted into this clamp frame 22a are biased in directions away from each other by the spring 22c. It should be noted that the length of a region of the clamp frame 22b inserted into the clamp frame 22a is more preferably made longer, so that the clamp frame 22a will not deflect due to the weight of the transfer membrane, its own weight, etc.

In addition, as shown in FIG. 10(b), in the clamp 25, fixing frames 28 are provided at three locations for each of the front clamp 27 and rear clamp 26, and the framed transfer membrane 10′ is fixed to the clamp 25 by fitting the ends of these fixing frames 28 into the fitting parts provided at three locations of the frames 2′ of the framed transfer membrane 10′. By the pair of frames 2′ being biased in directions away from each other by the spring 22c provided to the clamp frame 22′, tension is thereby acting on transfer membrane 1 of the framed transfer membrane 10′ so as to be tightened without slack, in a state retained by the clamp 25.

In addition, the clamp frame 22′ fixes the front clamp 27 and rear clamp 26 in a state separated by a certain distance. When fixing the framed transfer membrane 10′ by the clamp 25, the clamp 25 thereby fixes the front clamp 27 and rear clamp 26 from lateral sides relative to the conveying direction of the transfer membrane 1 (outer sides of two sides not supported by the frame 2′). When fixing the framed transfer membrane 10′ via the clamp 25 to the biomolecule analyzer 101, it is thereby possible to arrange so that the clamp frame 22′ does not contact the electrophoresis gel chip 50, or the guides 33 and 34, in the conveying path of the transfer membrane 1.

It should be noted that, as shown in FIG. 11, the clamp 25 is installed to the biomolecule analyzer 101 by fixing the front clamp 27 to the guide poles 66.

### (Operation of Biomolecule Analyzer 101)

An outline of a state mounting the framed transfer membrane 10′ to the biomolecule analyzer 101 will be explained using FIGS. 10 and 11. FIG. 11 is a view illustrating an outline of the biomolecule analyzer 101, and is a view illustrating a state installing the framed transfer membrane 10′ to the biomolecule analyzer 101, in a state of retaining the framed transfer membrane 10′ by the clamp 25. As shown in FIGS. 10(a) and (b), the framed transfer membrane 10′ fixes one of the frames 2′ to the front clamp 27, by causing a fitting part of the frame 2′ to fit with an end of the fixing frame 28 provided to the front clamp 27. In addition, in a similar way, it is fixed by causing the fitting part of the other frame 2′ to fit with an end of the fixing frame 2 provided to the rear clamp 26. The framed transfer membrane 10′ is thereby fixed by the clamp 25, as shown in FIG. 10(b).

Subsequently, as shown in FIG. 11, the framed transfer membrane 10′ fixed via the fixing frame 28 to the clamp 25 is arranged at the inner side of the anode buffer tank 30 filled with anode buffer, by fixing the front clamp 27 to the guide poles 66. As shown in FIG. 11, it is thereby possible to arrange the framed transfer membrane 10′ at the inner side of the anode buffer tank 30, by configuring so as not to immerse the clamp frame 22′ of the clamp 25 in the anode buffer tank 30. In addition, as shown in FIG. 11, the transfer membrane 1 of the framed transfer membrane 10′ is supported from the lower side by the guide 33 and guide 34, at an inner side of the anode buffer tank 30. Subsequently, similarly to the case of the biomolecule analyzer 100, the cathode buffer tank 40 to which the electrophoresis gel chip 50 is fixed by the lock 42 is fixed to the top part of the anode buffer tank 30. The transfer membrane 1 of the framed transfer membrane 10′ is thereby fixed in a state bent so as to become convex (valley folded shape) to an opposite side to the electrophoresis gel chip 50, by contacting the guide 33, guide 34 and electrophoresis gel chip 50.

Next, while being conveyed in the X direction shown in FIG. 11, in a state still pushing against the dispensing part of the electrophoresis gel chip 50, the transfer membrane 1 of the framed transfer membrane 10′r is transferred the analyte dispensed by the electrophoresis gel chip 50 by applying voltage between the anode 32 and cathode 41 in the anode buffer.

With the biomolecule analyzer 101, the transfer membrane 1 is imparted tension so as to be tightened without slack, by the spring 22c equipped to the clamp frame 22′ of the clamp 25. For this reason, it is possible to cause the tension acting on the transfer membrane 1 to focus on the dispensing part 50a provided at the end of the electrophoresis gel chip 50, while tension is applied to the transfer membrane 1 more downstream in the X direction than a portion pushed by the dispensing part 50a, by way of the biasing force of the spring 22c provided to the clamp frame 22′. For this reason, it is possible to prevent slack from arising in the transfer membrane 1 downstream from the portion pushed by the dispensing part 50a at which the tension on the transfer membrane 1 focuses. In other words, when conveying the transfer membrane 1 in the X direction, it is possible to prevent an interval between the transfer membrane 1 and dispensing part 50a from being able to form, by the transfer membrane 1 loosening on a downstream side of a region pushed by the dispensing part 50a of the electrophoresis gel chip 50. It is thereby possible to more suitably suppress the analyte dispensing from the dispensing part of the electrophoresis gel chip 50 from scattering in the anode buffer prior to being transferred to the transfer membrane 1. In addition, it is possible to more suitably reduce the fluctuation in the bands of analyte transferred to the transfer membrane 1, and thus it is possible to improve the sensitivity of the biomolecule analyzer.

### <Shaker (Shaker Device) 70>

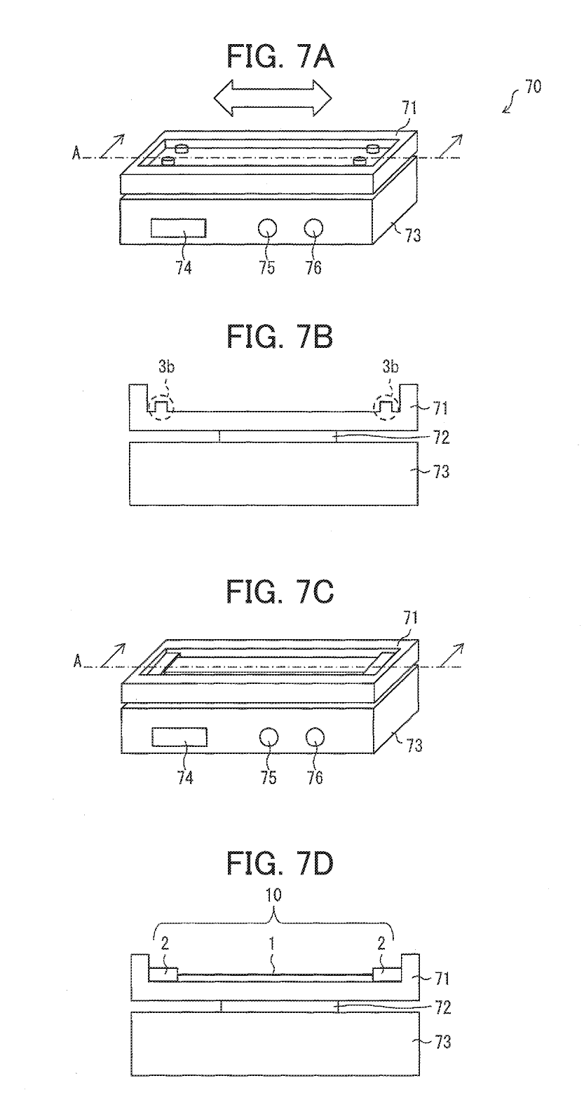

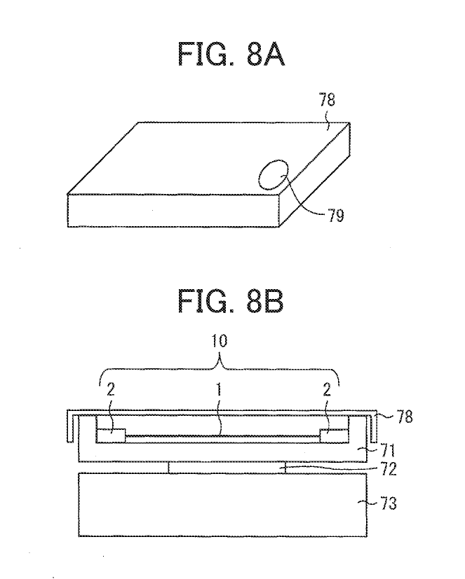

A shaker (shaker device) 70 according to one embodiment of the present invention will be explained in detail using FIGS. 7 and 8. FIG. 7(a) is a view illustrating an outline of the shaker according to the present embodiment, and FIG. 7(b) is a schematic view of a cross-section of the shaker along the line A.

As shown in FIGS. 7(a) and (b), the shaker 70 according to the present embodiment includes a reagent tank 71, drive unit 72 and shaker main body 73, and causes the reagent tank 71 to shake in the horizontal direction by way of the drive unit 72.

As shown in FIG. 7(b), the fitting part 3b, which can fit with the fitting part 3a of the framed transfer membrane 10, is provided to an inner side of the reagent tank 71. It is thereby possible to fit the fitting part 3a of the framed transfer membrane 10 to the fitting part 3b, and fix the transfer membrane 1 to the inner side of the reagent tank 71 (FIGS. 7(c) and (d)).

As shown in FIG. 7(c), the framed transfer membrane 10 mounted at the inner side of the reagent tank 71 is fixed in a state in which the transfer membrane 1 is spread evenly. For this reason, when carrying out antibody response on the analyte transferred to the transfer membrane 1, it is possible to prevent the transfer membrane 1 from moving in the reagent tank 71. For this reason, it is possible to more uniformly carry out antibody response on analytes such as proteins transferred to the transfer membrane 1.

In addition, as shown in FIGS. 8(a) and (b), in the case of marking the proteins transferred to the transfer membrane 1 using the reagent tank 71, the shaker 70 more preferably includes a shading lid 78 that shades the inner side of the reagent tank 71. A window 79 that can expose light onto the modified region 8 of the frame 2′ arranged at the inside is provided to the shading lid 78.

By modifying the modified region 8 with a standard antibody, etc. for confirming the progress of antibody response, it is thereby possible to maintain the shaded state for the transfer membrane 1 fixed in the reagent tank 71, and possible to expose light to the modified region 8 provided to the frame 2′. For this reason, it is possible to confirm the state of progress of antibody response, by confirming the fluorescence intensity of the modified region 8.

It should be noted that, as shown in FIG. 7(a), it is preferable for a display unit 74 that displays a time of the transfer membrane, revolution speed of the drive unit 72, etc., and adjusting parts 75 and 76 for adjusting the shaker time and revolution speed, etc. to be provided to the shake main body 73.

The present invention is not to be limited to the aforementioned respective embodiments, with various modifications within the scope shown in the claims being possible, and embodiments achieved by appropriately combining the technical means disclosed in each of the different embodiments are also included in the technical scope of the present invention. Furthermore, it is possible to form novel technical features by combining the technical means disclosed in each of the respective embodiments.

In the above way, a transfer membrane with frame member according to a first aspect of the present invention includes: a transfer membrane to which an analyte separated by way of electrophoresis is transferred; and a pair of frame members that individually support two sides, which are a first side that is one side of the transfer membrane, and a second side that is a side thereof on a side opposing the first side.

According to the above-mentioned configuration, it is possible to easily retain the transfer membrane via the pair of frame members. In addition, since the pair of frame members individually support the two sides of the transfer membrane opposing each other, it is possible to tighten the transfer membrane without slack, by retaining by allowing the frame members to be separated. Furthermore, since the frame members are not provided to the transfer membrane other than at the first side and second side thereof, processing after transferring will not be inhibited as in the curved frame of Patent Document 1. According to the above-mentioned transfer membrane with frame member, it is possible to easily tighten the transfer membrane without slack in this way, without inhibiting processing after transfer.

In the transfer membrane with frame member according to a second aspect of the present invention, fitting parts for fitting with an external member may be formed in each of the pair of frame members.

According to the above-mentioned configuration, by fitting the fitting part of the frame member with an external member, it is possible to more easily retain the transfer membrane via the frame member.

In the transfer membrane with frame member according to a third aspect of the present invention, the fitting part for fitting with the external member may be a through-hole, recess (concave part) or projecting part.

According to the above-mentioned configuration, it is possible to easily fit the fitting part of the frame member with an external member.

In the transfer membrane with frame member according to a fourth aspect of the present invention, the frame member includes a modified region that is modified by a fluorescence reference sample.

According to the above-mentioned configuration, it is possible to perform fluorescence detection processing on analyte transferred to the transfer membrane, based on the fluorescence intensity emitted by the fluorescence reference sample modifying the modified region.

In the transfer membrane with frame member according to a fifth aspect of the present invention, one frame member among the pair of frame members pinches the first side, and another of the frame members pinches the second side, thereby individually supporting the two sides of the transfer member.

According to the above-mentioned configuration, it is possible to suitably support the transfer membrane by the frame member.

In the transfer membrane with frame member according to a sixth aspect of the present invention, each of the pair of frame members includes a first face and a second face forming a pair to pinch the transfer membrane, respectively; the first face includes a ridge part having a profile of a cross-section in a short direction that is a curved line; and the second face includes a grooved part that fits with the ridge part.

According to the above-mentioned configuration, it is possible to raise the frictional force at the first face and second face of the frame member when sandwiching the transfer membrane. For this reason, when conveying the framed transfer membrane in the biomolecule analyzer, it is possible to suitably prevent the transfer membrane from detaching from the frame of the framed transfer membrane.

In the transfer membrane with frame member according to a seventh aspect of the present invention, the frame member includes an entry region in which entry of information is possible.

According to the above-mentioned configuration, it is possible to record various information such as the type of analyte, evaluation conditions, and date on which transfer was performed, for every transfer membrane with frame member.

In the transfer membrane with frame member according to an eighth aspect of the present invention, the pair of frame members is subjected to hydrophilization treatment on a surface.

According to the above-mentioned configuration, it is possible to prevent proteins, antibodies or the like from adhering to the frame member. Therefore, it is possible to prevent the frame member from contaminating.

In the transfer membrane with frame member according to a ninth aspect of the present invention, it is preferable for the pair of frame members to consist of an insulating material.

According to the above-mentioned configuration, it is possible to prevent the line of electric force from changing due to the frame member upon performing electrophoresis using the transfer membrane with frame member.

A biomolecule analyzer according to a tenth aspect of the present invention includes: a transfer member with frame member according to according to an aspect of the present invention; a retaining part that retains the transfer membrane with frame member; a conveying unit that conveys the retaining part along a conveying direction that is parallel to a direction from the first side towards the second side; and a separation unit that is installed so as to vertically abut against the transfer membrane, separates an analyte by way of electrophoresis, and dispenses the analyte thus separated onto the transfer membrane, in which the retaining part includes a pair of fixing parts to which the pair of frame members is fixed, respectively.

According to the above-mentioned configuration, the retaining part can easily tighten the transfer membrane without slack, by fixing the pair of frame members to each of the different fixing parts. It is thereby possible to transfer the analyte dispensed from the separation unit to the transfer membrane with good sensitivity.

In the biomolecule analyzer according to an eleventh aspect of the present invention, the pair of fixing parts each includes a biasing part that biases the pair of frame members in directions away from each other.

According to the above-mentioned configuration, it is possible to prevent slack from arising in the transfer membrane, more downstream in the conveying direction than a portion pressed by the separation unit of the biomolecule analyzer against the transfer membrane.

In the biomolecule analyzer according to a twelfth aspect of the present invention, the frame member and the fixing part include fitting parts for fitting with each other, and the frame member is fixed to the fixing part by way of the fitting parts.

According to the above-mentioned configuration, it is possible to more suitably fix the transfer membrane with frame member and the retaining part via the frame member.

The biomolecule analyzer according to a thirteenth aspect of the present invention further includes: a support member that supports, from an opposite side of the transfer membrane than the separation unit, a pair of positions of the transfer membrane that interpose from front and rear in the conveying direction, a position abutted by the separation unit, in which the transfer membrane is bent to become convex to an opposite side than the separation unit, by abutting the separation unit in a state supported by the support member.

According to the above-mentioned configuration, it is possible to install in state such that the separation unit is pressed against the transfer membrane of the transfer membrane with frame member from an upper side. In addition, the transfer member is bent so as to become convex (valley folded shape) to an opposite side than the separation unit, by contacting the support members provided at two locations and the separation unit. It is thereby possible for tension to act on the transfer membrane to cause the transfer membrane to closely contact the separation unit. It is thereby possible to more suitably perform transfer from the separation medium to the transfer membrane.

In particular, according to the above-mentioned configuration, it is possible to easily impart tension to the transfer membrane due to retaining the transfer membrane via the frame member.

In a reagent tank according to a fourteenth aspect of the present invention for immersing the transfer membrane with frame member according to the second or third aspect in a reagent for antibody response, a fitting part for fitting with the fitting part of the frame member is provided at an inner side of the reagent tank.

According to the above-mentioned such configuration, since it is possible to prevent the transfer membrane from turning over or overlapping, the antibody response can be uniformly carried out on (analyte transferred to) the transfer membrane.

In the reagent tank according to a fifteenth aspect of the present invention, the reagent tank further includes a shading lid that shades the inside of the reagent tank, and a window disposed above the frame member is provided to the shading lid.

According to the above-mentioned configuration, it is possible to confirm above the frame membrane from a window while shading by the shading lid. Herein, by modifying with a standard antibody, etc. for confirming the progress of antibody response on the frame member, it is possible to confirm the state of progress of the antibody response, without inhibiting the reaction on the transfer membrane.

A shaker device according to a sixteenth aspect of the present invention includes: a reagent tank according to the fourteenth or fifteenth aspect; and a drive unit for causing the reagent tank to shake.

According to the above-mentioned configuration, it is possible to uniformly carry out the antibody response on analyte transferred to the transfer membrane.

### INDUSTRIAL APPLICABILITY

The present invention can be suitably applied to a two-dimensional electrophoresis apparatus.

### EXPLANATION OF REFERENCE NUMERALS

1 transfer membrane
2, 2′, 4, 6, 6′ frame (frame member)
3a fitting part
3b fitting part
5a, 5b fitting part
7a, 7′a fitting part
8 modified region
9a fitting part
10, 11, 12, 12′ framed transfer membrane
20, 25 clamp (retaining part)
22, 22′ clamp frame (fixing part)
33 guide (support member)
34 guide (support member)
50 electrophoresis gel chip (separation unit)
62 motor (conveying unit)
63 ball screw (conveying unit)
64 guide shaft (conveying unit)
65 shaft holder (conveying unit)
66 guide pole (conveying unit)
70 shaker (shaker device)
72 drive unit
78 shading lid
79 window
100 biomolecule analyzer
101 biomolecule analyzer

1. A transfer membrane with frame member, comprising:
a transfer membrane to which an analyte separated by way of electrophoresis is transferred; and
a pair of frame members that individually support two sides, which are a first side that is one side of the transfer membrane, and a second side that is a side thereof on a side opposing the first side.

2. The transfer membrane with frame member according to claim 1, wherein fitting parts for fitting with an external member are formed in each of the pair of frame members.

3. The transfer membrane with frame member according to claim 2, wherein the fitting part for fitting with the external member is a through-hole, concave part or projecting part.

4. The transfer membrane with frame member according to claim 1, wherein the frame member includes a modified region that is modified by a fluorescence reference sample.

5. The transfer membrane with frame member according to claim 1, wherein one frame member among the pair of frame members pinches the first side, and another of the frame members pinches the second side, thereby individually supporting the two sides of the transfer member.

6. The transfer membrane with frame member according to claim 5, wherein each of the pair of frame members includes a first face and a second face forming a pair to pinch the transfer membrane, respectively,
wherein the first face includes a ridge part having a profile of a cross-section in a short direction that is a curved line, and
wherein the second face includes a grooved part that fits with the ridge part.

7. The transfer membrane with frame member according to claim 1, wherein the frame member includes an entry region in which entry of information is possible.

8. The transfer membrane with frame member according to claim 1, wherein the pair of frame members is subjected to hydrophilization treatment on a surface.

9. The transfer membrane with frame member according to claim 1, wherein the pair of frame members consists of an insulating material.

10. A biomolecule analyzer comprising:
a transfer member with frame member according to claim 1;
a retaining part that retains the transfer membrane with frame member;
a conveying unit that conveys the retaining part along a conveying direction that is parallel to a direction from the first side towards the second side; and
a separation unit that is installed so as to vertically abut against the transfer membrane, separates an analyte by way of electrophoresis, and dispenses the analyte thus separated onto the transfer membrane,
wherein the retaining part includes a pair of fixing parts to which the pair of frame members is fixed, respectively.

11. The biomolecule analyzer according to claim 10, wherein the pair of fixing parts each includes a biasing part that biases the pair of frame members in directions away from each other.

12. The biomolecule analyzer according to claim 10 wherein the frame member and the fixing part include fitting parts for fitting with each other, and the frame member is fixed to the fixing part by way of the fitting parts.

13. The biomolecule analyzer according to claim 10, further comprising a support member that supports, from an opposite side of the transfer membrane than the separation unit, a pair of positions of the transfer membrane that interpose a position abutted by the separation unit from front and rear in the conveying direction,
wherein the transfer membrane is bent to become convex to an opposite side than the separation unit, by abutting the separation unit in a state supported by the support member.

14. A reagent tank for immersing the transfer membrane with frame member according to claim 2 in a reagent for antibody response,
wherein a fitting part for fitting with the fitting part of the frame member is provided at an inner side of the reagent tank.

15. The reagent tank according to claim 14, wherein the reagent tank further comprises a shading lid that shades the inside of the reagent tank, and
wherein a window disposed above the frame member is provided to the shading lid.

16. A shaker device comprising a reagent tank according to claim 14, and a drive unit for causing the reagent tank to shake.
